(19) United States
(12) Patent Application Publication
(10) Pub. No.: US 2019/0154703 A1
(43) Pub. Date: May 23, 2019

(54) Marker for Prognosis of a Clinical Outcome of an Autologous Intervertebral Disc Cell Transplantation, for Progress Assessment/Progress Monitoring of an Autologous Intervertebral Disc Cell Transplantation, for Assessing the Quality of Intervertebral Disc Cells, for Assessing the Quality of an Implant and/or Medication for Novel Therapies (ATMP) for the Treatment of an Intervertebral Disc Defect, and for Diagnosis of an Intervertebral and/or Spinal Column Defect

(71) Applicant: TETEC TISSUE ENGINEERING TECHNOLOGIES AG, Reutlingen (DE)

(72) Inventors: Christoph GAISSMAIER, Kusterdingen (DE); Karin BENZ, Göppingen (DE); Bernd ROLAUFFS, Tübingen (DE)

(73) Assignee: TETEC Tissue Engineering Technologies AG, Reutlingen (DE)

(21) Appl. No.: 16/097,598

(22) PCT Filed: Apr. 25, 2017
(86) PCT No.: PCT/EP2017/059776

§ 371 Date: Oct. 29, 2018

(30) Foreign Application Priority Data
Apr. 29, 2016 (DE) 10 2016 207 462.8

(51) Int. Cl. G01N 33/68

(52) U.S. Cl. CPC G01N 33/6887; G01N 2800/10; G01N 2333/78

(57) ABSTRACT

The invention relates to markers for use in vitro in the prognosis of a clinical outcome (outcome prognosis) of an autologous disc cell transplantation, in the progress assessment/progress monitoring of an autologous disc cell transplantation, in the assessment of the quality of intervertebral disc cells, in the assessment of the quality of an implant and/or advanced therapy medicinal products (ATMP) for the treatment of an intervertebral disc defect, and/or in the diagnosis of an intervertebral disc and/or spinal column defect and a corresponding in vitro method.

### FIELD OF APPLICATION AND PRIOR ART

The invention relates to markers for use in vitro in the prognosis of a clinical outcome (outcome prognosis) of an autologous disc cell transplantation, in the progress assessment/progress monitoring of an autologous disc cell transplantation, in the assessment of the quality of intervertebral disc cells, in the assessment of the quality of an implant and/or advanced therapy medicinal products (ATMP) for the treatment of an intervertebral disc defect, and/or in the diagnosis of an intervertebral disc and/or spinal column defect and a corresponding in vitro method.

Degeneration of the intervertebral discs is one of the primary causes of back pain, which not only causes major suffering to individual patients, but also constitutes a worldwide socioeconomic burden.

The etiology and pathogenesis of intervertebral disc degeneration have not been fully clarified. The pathological degeneration process appears to be connected to varying degrees with the shock-absorbing portion of the intervertebral disc, namely its gelatinous core (the nucleus pulposus), its protective fiber ring (the annulus fibrosus), and the vertebral end plates.

Following an initial event such as trauma or overloading, inflammatory processes initially come to the fore, leading to various biochemical and morphological changes in the intervertebral disc. These include the loss of extracellular matrix proteins, such as for example water-binding proteoglycans, the resulting secondary drying of the intervertebral disc core with a decrease in tissue pH, and the death of intervertebral disc cells with increasing development of a catabolic metabolic state. In the course of degeneration, moreover, pathological neoangiogenesis and neurogenesis (painful disc) occur, as well as scar tissue formation in the intervertebral disc accompanied by ossification.

These morphological changes affect the core of the intervertebral disc, and in the further course of the process fiber ring as well. In image diagnosis, this appears among other signs as a decrease in intervertebral disc height (caused in particular by dehydration). As a result, one can expect reduced tension of the annular fibers between the adjacent vertebrae.

Moreover, on histological and MRT examination, fissures in the fiber ring (high-intensity zones) are observed, with pathological disruptions of the annular lamellae and reduced vascularization of the end plates. All of these changes combine to cause impaired shock-absorbing properties of the core, reduced tensile strength of the fiber ring, and impaired nutrient supply to the intervertebral disc.

The decrease in intervertebral disc height promotes the onset of segmental instability, which is thought to further accelerate segment degeneration due to microtrauma. If the annulus fibrosus tears, a slipped intervertebral disc can occur, which depending on its location, size, and consistency may lead to pressure irritation of nerves, as well as considerable pain and functional impairment resulting from inflammatory reactions. In the event of significant neurological deficits, surgical repair of the slipped disc may be required.

If conservative treatment is impossible, or once conservative treatment possibilities have been exhausted, invasive treatment methods are used.

A possible invasive treatment of slipped intervertebral discs is so-called sequestrectomy or nucleotomy. In this procedure, protruding intervertebral disc fragments are removed from the spinal or spinal nerve canal. The problem is that a sequestrectomy or nucleotomy often does not leave the patient symptom-free.

A further treatment consists in carrying out so-called spondylodesis, i.e. spinal fusion. This is a surgical procedure in which the section of the spinal column to be fused is immobilized using a combination of screws and small connecting rods. Depending on the extent of the procedure, surgical fusion usually produces a favorable outcome. Over a period of years, however, the result is impaired by so-called connection instability. In this case, adjacent vertebral segments are unduly strained after fusion and in turn cause back pain. As a rule, therefore, long-term freedom from symptoms cannot be expected.

In complete removal of the intervertebral disc, it is generally necessary to insert an implant that acts as a placeholder.

A further treatment method is so-called disc cell transplantation (ADCT, autologous disc-derived chondrocyte transplantation). In this treatment modality, usually in the context of an acute slipped intervertebral disc, the intervertebral disc tissue that protrudes through the outer fiber ring is surgically removed. By subsequent mechanical size reduction and enzymatic digestion, the intervertebral disc cells contained in the removed intervertebral disc tissue are dissolved and expanded in vitro under sterile conditions, i.e. propagated. After the required cell count is reached, the intervertebral disc cells can be “harvested” and inserted into the remaining intervertebral disc core. The inserted intervertebral disc cells colonize the defect area, and in the further course of the process, begin to produce cartilage ground substance.

An improved form of autologous disc cell transplantation is so-called matrix-associated disc cell transplantation. In this method, the intervertebral disc cells are used in combination with a suitable matrix, i.e. in a carrier material.

However, a drawback of autologous disc cell transplantation is that the autologous intervertebral disc cells can vary widely depending on the patient, which can strongly affect the therapeutic course of autologous disc cell transplantation. Moreover, the intervertebral disc cells may undergo changes in their properties during their in vitro expansion, which can also affect the course of autologous disc cell transplantation.

Of course, implants or carrier materials for intervertebral disc cells used in the treatment of intervertebral disc defects also have a decisive effect on the treatment outcome.

### OBJECT AND MEANS FOR ACHIEVING OBJECT

Against this backdrop, the object of the invention is to provide markers that allow the prediction of a clinical outcome of an autologous disc cell transplantation, in particular a matrix-associated autologous disc cell transplantation.

A further object of the invention is to provide markers that allow an assessment of the quality of intervertebral disc cells.

A further object of the invention is to provide markers that allow an assessment of the quality of implants and/or advanced therapy medicinal products (ATMPs) for the treatment of an intervertebral disc defect.

A further object of the invention is to provide markers that allow the diagnosis of an intervertebral disc and/or spinal column defect.

An additional object of the invention is to provide a corresponding in vitro method and use of a kit for carrying out the in vitro method.

These objects are achieved according to the invention described herein. The wording of all of the claims is hereby incorporated into the contents of the present description by express reference.

The invention relates to a marker for use in vitro
in the prediction (prognosis) of a clinical outcome (so-called outcome prognosis) of an autologous disc cell transplantation, in particular a matrix-associated autologous disc cell transplantation,
   and/or
in the progress assessment/progress monitoring of an autologous disc cell transplantation, in particular a matrix-associated autologous disc cell transplantation,
   and/or
in the assessment of the quality of intervertebral disc cells, i.e. so-called intervertebral disc chondrocytes, preferably for an autologous disc cell transplantation, in particular for a matrix-associated autologous disc cell transplantation,
   and/or
in the assessment of the quality of an implant and/or advanced therapy medicinal products (ATMP) for the treatment of an intervertebral disc defect and/or for the reconstruction of intervertebral disc tissue,
   and/or
in the diagnosis of an intervertebral disc defect, in particular a lumbar, preferably lumbar-sacral, intervertebral disc defect, and/or a spinal column defect,
   wherein the marker is selected from the group consisting of CTX-I, CTX-II, NTX-I, CPII, VEGF, C2C, MMP-3, hyaluronic acid, TNF-α, IL-1ra, IL-4, COMP, YKL-40 and combinations of at least two of said markers.

The term “marker” can therefore refer in the context of the present invention to one of the above-mentioned markers or a combination of two or more, i.e. a combination of two, three, four, five, six, seven, eight, nine, ten, eleven, or twelve of the above-mentioned markers or a combination of all of the above-mentioned markers.

Preferably, the term “marker” refers in the context of the present invention to the corresponding marker protein or the corresponding combination of marker proteins. In other words, the term “marker” in the context of the present invention preferably refers to a marker protein selected from the group consisting of CTX-I, CTX-II, NTX-I, CPII, VEGF, C2C, MMP-3, hyaluronic acid, TNF-α, IL-1ra, IL-4, COMP, YKL-40 and combinations of two or more of said proteins.

The term “prediction of a clinical outcome” is to be understood in the context of the present invention as referring to the prognosis of a clinical outcome that will probably occur.

The term “prediction of a positive clinical outcome” is to be understood in the context of the present invention as referring to the prognosis of a clinical improvement compared to the clinical status before treatment or therapy was begun that will probably occur.

The term “prediction of a negative clinical outcome” is to be understood in the context of the present invention as referring to the prognosis of a clinical deterioration compared to the clinical status before treatment or therapy was begun that will probably occur.

The term “quality of the intervertebral disc cells” is to be understood in the context of the present invention as referring to a protein synthesis performance determined at the end of in vitro cultivation of intervertebral disc cells.

The term “positive assessment of the quality of the intervertebral disc cells” is to be understood in the context of the present invention as referring to a protein synthesis performance that is sufficient for providing an anti-inflammatory action and/or a neosynthesis of extracellular matrix and/or a functional improvement of a treated intervertebral disc.

The term “negative assessment of the quality of the intervertebral disc cells” is to be understood in the context of the present invention as referring to a protein synthesis performance that is insufficient for providing an anti-inflammatory action and/or a neosynthesis of extracellular matrix and/or a functional improvement of a treated intervertebral disc.

The term “quality of an implant and/or advanced therapy medicinal products (ATMP)” is to be understood in the context of the present invention as referring to the effect of an implant and/or advanced therapy medicinal products on inflammation/pain and/or tissue regeneration and/or functional improvement of a treated intervertebral disc cell.

The term “positive assessment of the quality of an implant and/or advanced therapy medicinal products (ATMP)” is to be understood in the context of the present invention as referring to the positive effect of an implant and/or medication on inflammation/pain and/or tissue regeneration and/or functional improvement of a treated intervertebral disc.

The term “negative assessment of the quality of an implant and/or advanced therapy medicinal products (ATMP)” is to be understood in the context of the present invention as referring to the negative or lacking effect of an implant and/or medication on inflammation/pain and/or tissue regeneration and/or functional improvement of a treated intervertebral disc.

The term “intervertebral disc cells” is to be understood in the context of the present invention as referring to intervertebral disc cartilage cells, i.e. so-called intervertebral disc chondrocytes.

The term “treatment of an intervertebral disc defect” is to be understood in the context of the present invention as referring in particular to an autologous disc cell transplantation, such as in particular a matrix-associated autologous disc cell transplantation, an implant-based intervertebral disc treatment, or an intervertebral disc treatment based on advanced therapy medicinal products (ATMP). Preferably, the term “treatment of an intervertebral disc defect” refers in the context of the present invention to an autologous disc cell transplantation, in particular a matrix-associated autologous disc cell transplantation.

The term “autologous disc cell transplantation” is to be understood in the context of the present invention as referring to the transplantation of autologous intervertebral disc cells (intervertebral disc chondrocytes) into a damaged intervertebral disc. The transplantation preferably takes place by injection. With respect to further details, reference is made to the explanations given above.

The term “matrix-associated autologous disc cell transplantation” is to be understood in the context of the present invention as referring to the transplantation of autologous intervertebral disc cells (intervertebral disc chondrocytes), wherein the autologous intervertebral disc cells are inserted together with a suitable matrix, i.e. a carrier material, into a damaged intervertebral disc between two adjacent vertebrae, in particular for the reconstruction of intervertebral disc tissue. The matrix or the carrier material can for example be a hydrogel or components for the production of a hydrogel. Preferably, the matrix or the carrier material is a two-component system for the production of a hydrogel or a hydrogel produced therefrom, wherein one component of the system comprises maleimide-functionalized albumin and hyaluronic acid and the other component comprises bis-thio-polyethylene glycol (bis-thio-PEG). On mixing of the components, for example on dispensing from a two-compartment syringe, a crosslinking reaction takes place between the maleimide-functionalized albumin and the bis-thio-polyethylene glycol, resulting in the formation of a hydrogel.

The term “advanced therapy medicinal products (ATMP)” is to be understood in the context of the present invention as referring to tissue-engineering products with autologous intervertebral disc cells and biocompatible carrier materials for the autologous intervertebral disc cells. An example of an advanced therapy medicinal product (ATMP) is a two-component system for the production of a hydrogel or a hydrogel produced therefrom, wherein one component of the system comprises maleimide-functionalized albumin, autologous intervertebral disc cells and hyaluronic acid and the other component of the system comprises bis-thio-polyethylene glycol (bis-thio-PEG). An example of such an ATMP is described in further detail in the example section of the present application under the name “Novocart® Disc plus”.

The term “implant-based intervertebral disc treatment” is to be understood in the context of the present invention as referring to the treatment of an intervertebral disc defect based on an implant, and preferably without the use of intervertebral disc cells.

The term “intervertebral disc defect” is to be understood in the context of the present invention as referring in particular to an intervertebral disc prolapse or an intervertebral disc extrusion.

The term “intervertebral disc prolapse,” also referred to as a disc prolapse or slipped intervertebral disc, is to be understood in the context of the present invention as referring to a disorder of the spinal column in which parts of the intervertebral disc, in particular of the gelatinous intervertebral disc core (nucleus pulposus), protrude into the spinal canal, i.e. the space containing the spinal cord, wherein the fibrous cartilage ring (annulus fibrosus) of the intervertebral disc—in contrast to intervertebral disc extrusion—is completely or partially torn, while the posterior longitudinal ligament (ligamentum longitudinale posterius) may remain intact (a so-called subligamental slipped intervertebral disc).

The term “intervertebral disc tissue” is to be understood in the context of the present invention as referring to the gelatinous core of the intervertebral disc, i.e. the so-called nucleus pulposus, and/or the fiber ring of the intervertebral disc, i.e. the so-called annulus fibrosus. Preferably, the term “intervertebral disc tissue” within the meaning of the present invention defines the gelatinous core of the intervertebral disc, i.e. the nucleus pulposus.

The term “treated intervertebral disc” is to be understood in the context of the present invention as referring to an intervertebral disc that was preferably treated by means of an autologous disc cell transplantation, such as in particular a matrix-assisted autologous disc cell transplantation, an implant, or advanced therapy medicinal products.

The term “increased concentration” refers in the context of the present invention, unless otherwise described below, to a concentration of the marker or markers in question in a body sample taken after treatment of an intervertebral disc defect that is increased compared to the concentration of the corresponding marker or markers in a body sample taken before treatment of the intervertebral disc defect, in particular prior to a sequestrectomy.

The term “decreased concentration” refers in the context of the present invention, unless otherwise described below, to a concentration of the marker or markers in question in a body sample taken after treatment of an intervertebral disc defect that is decreased compared to the concentration of the corresponding marker or markers in a body sample taken before treatment of the intervertebral disc defect, in particular prior to a sequestrectomy.

The terms “determine” and “test” are to refer in the context of the present invention to any biochemical and/or biotechnological method by means of which the concentration of the marker or markers in question can be identified in one or a plurality of body samples, in particular over the course of time (i.e. in two or more body samples taken from a patient at different times).

The term “body sample” is to be understood in the context of the present invention as referring to a sample taken from a patient.

The term “patient” is to be understood in the context of the present invention as referring to a human or animal patient, preferably a human.

In a preferred embodiment, the marker is CTX-I, also referred to a a-CTX. CTX-I is the C-terminal telopeptide of collagen type I. The telopeptide is released in the breakdown of collagen type I.

In particular, the marker can be a combination of CTX-I and at least one further marker selected from the group consisting of CTX-II, NTX-I, CPII, VEGF, C2C, MMP-3, hyaluronic acid, TNF-α, IL-1ra, IL-4, COMP and YKL-40.

In a further embodiment, the marker is CTX-II. This is the C-terminal telopeptide of collagen type II. CTX-II is composed of six amino acids and is located in the areas of the non-helical carboxy terminal crosslinked telopeptides of collagen type II.

In particular, the marker can be a combination of CTX-II and at least one further marker selected from the group consisting of CTX-I, NTX-I, CPII, VEGF, C2C, MMP-3, hyaluronic acid, TNF-α, IL-1ra, IL-4, COMP and YKL-40.

In a further embodiment, the marker is NTX-I. NTX-I is an N-terminal telopeptide of collagen type I that is elevated in bone breakdown.

In particular, the marker can be a combination of NTX-I and at least one further marker selected from the group consisting of CTX-I, CTX-II, CPII, VEGF, C2C, MMP-3, hyaluronic acid, TNF-α, IL-1ra, IL-4, COMP and YKL-40.

In a further embodiment, the marker is CPII, also referred to as chondrocalcin. This is the carboxy terminal peptide group of procollagen type II.

In particular, the marker can be a combination of CPII and at least one further marker selected from the group consisting of CTX-I, CTX-II, NTX-I, VEGF, C2C, MMP-3, hyaluronic acid, TNF-α, IL-1ra, IL-4, COMP and YKL-40.

In a further embodiment, the marker is VEGF (vascular endothelial growth factor).

In particular, the marker can be a combination of VEGF and at least one further marker selected from the group consisting of CTX-I, CTX-II, NTX-I, CPII, C2C, MMP-3, hyaluronic acid, TNF-α, IL-1ra, IL-4, COMP and YKL-40.

In a further embodiment, the marker is C2C. This is a neoepitope occurring in the primary cleavage of collagen type II on the “new” carboxy terminal end of the longer (¾) cleavage product. Further cleavage gives rise to a fragment composed of approximately 45 amino acid moieties that contains the C2C epitope.

In particular, the marker can be a combination of C2C and at least one further marker selected from the group consisting of CTX-I, CTX-II, NTX-I, CPII, VEGF, MMP-3, hyaluronic acid, TNF-α, IL-1ra, IL-4, COMP and YKL-40.

In a further embodiment, the marker is MMP-3 (matrix metalloproteinase-3).

In particular, the marker can be a combination of MMP-3 and at least one further marker selected from the group consisting of CTX-I, CTX-II, NTX-I, CPII, VEGF, C2C, hyaluronic acid, TNF-α, IL-1ra, IL-4, COMP and YKL-40.

In a further embodiment, the marker is hyaluronic acid.

In particular, the marker can be a combination of hyaluronic acid and at least one further marker selected from the group consisting of CTX-I, CTX-II, NTX-I, CPII, VEGF, C2C, MMP-3, TNF-α, IL-1ra, IL-4, COMP and YKL-40.

In a further embodiment, the marker is TNF-α (tumor necrosis factor-a).

In particular, the marker can be a combination of TNF-α and at least one further marker selected from the group consisting of CTX-I, CTX-II, NTX-I, CPII, VEGF, C2C, MMP-3, hyaluronic acid, IL-1ra, IL-4, COMP and YKL-40.

In a further embodiment, the marker is IL-1ra (interleukin-1 receptor antagonist).

In particular, the marker can be IL-1ra and at least one further marker selected from the group consisting of CTX-I, CTX-II, NTX-I, CPII, VEGF, C2C, MMP-3, hyaluronic acid, TNF-α, IL-4, COMP and YKL-40.

In a further embodiment, the marker is IL-4 (interleukin-4).

In particular, the marker can be a combination of IL-4 and at least one further marker selected from the group consisting of CTX-I, CTX-II, NTX-I, CPII, VEGF, C2C, MMP-3, hyaluronic acid, TNF-α, IL-1ra, COMP and YKL-40.

In a further embodiment, the marker is COMP (cartilage oligomeric matrix protein).

In particular, the marker can be a combination of COMP and at least one further marker selected from the group consisting of CTX-I, CTX-II, NTX-I, CPII, VEGF, C2C, MMP-3, hyaluronic acid, TNF-α, IL-1ra, IL-4 and YKL-40.

In a further embodiment, the marker is YKL-40 (chitinase-3-like protein 1) (CHI3L1)).

In particular, the marker can be a combination of YKL-40 and at least one further marker selected from the group consisting of CTX-I, CTX-II, NTX-I, CPII, VEGF, C2C, MMP-3, hyaluronic acid, TNF-α, IL-1ra, IL-4 and COMP.

According to a particularly preferred embodiment, the marker is selected from the group consisting of CTX-I, CTX-II, NTX-I, CPII, C2C, VEGF, IL-1ra, MMP-3, and combinations of two or more of said markers, in particular from the group consisting of CTX-I, CTX-II, NTX-I, CPII, VEGF, MMP-3 and combinations of two or more of said markers.

In a further embodiment, the marker or markers is/are the protein in question or the proteins in question. In other words, the marker or markers is/are preferably a protein selected from the group consisting of CTX-I, CTX-II, NTX-I, CPII, VEGF, C2C, MMP-3, hyaluronic acid, TNF-α, IL-1ra, IL-4, COMP, YKL-40 and combinations of two or more of said proteins.

In a further embodiment, the concentration of the marker is determined in body samples taken from a patient at different times.

The plural term “body samples” refers in the context of the present invention to two body samples or multiple body samples, such as for example three, four or five body samples.

Preferably, an increase or decrease in the concentration of the marker over time, i.e. a concentration of the marker that increases or decreases as the body samples are taken, is tested. In other words, the concentration of the marker is preferably determined in each body sample taken, followed by testing to determine whether the concentrations determined increased or decreased according to the sequence over time in which the body samples were taken.

Moreover, an increase or decrease in the marker over time is preferably correlated with the prediction of a positive clinical outcome of an autologous disc cell transplantation, in particular a matrix-associated autologous disc cell transplantation, and/or with a positive progress assessment/progress monitoring of an autologous disc cell transplantation, in particular a matrix-associated autologous disc cell transplantation, and/or with a positive assessment of the quality of an implant and/or advanced therapy medicinal products (ATMP) for the treatment of an intervertebral disc defect and/or for the reconstruction of intervertebral disc tissue, or is correlated with the prediction of a negative clinical outcome of an autologous disc cell transplantation, in particular a matrix-associated autologous disc cell transplantation, and/or with a negative progress assessment/progress monitoring of an autologous disc cell transplantation, in particular a matrix-associated autologous disc cell transplantation, and/or with a negative assessment of the quality of an implant and/or advanced therapy medicinal products (ATMP) for the treatment of an intervertebral disc defect and/or for the reconstruction of intervertebral disc tissue.

According to a particularly preferred embodiment, the concentration of the marker is the protein concentration of the marker.

In a further embodiment, the body samples comprise
at least one body sample taken from the patient prior to a sequestrectomy or nucleotomy, and/or
at least one body sample taken from the patient after sequestrectomy and before an autologous disc cell transplantation, matrix-associated autologous disc cell transplantation, implant-based intervertebral disc treatment or an intervertebral disc treatment based on advanced therapy medicinal products, and/or
at least one body sample taken from the patient after an autologous disc cell transplantation, in particular a matrix-associated autologous disc cell transplantation, implant-based intervertebral disc treatment or an intervertebral disc treatment based on advanced therapy medicinal products.

The term “at least one body sample” can refer in the context of the present invention to one body sample or a plurality of body samples, such as for example two, three or four body samples.

The body samples can comprise in particular at least one body sample that was taken from the patient over a period of up to 24 months, in particular 12 months, after sequestrectomy, an autologous disc cell transplantation, such as in particular a matrix-associated autologous disc cell transplantation, implant-based intervertebral disc treatment, or an intervertebral disc treatment based on advanced therapy medicinal products.

The body samples can comprise in particular body samples taken from the patient 1.5 months, 3 months, 6 months, 12 months and/or 24 months after sequestrectomy, an autologous disc cell transplantation, such as in particular a matrix-associated autologous disc cell transplantation, implant-based intervertebral disc treatment, or an intervertebral disc treatment based on advanced therapy medicinal products.

In a further embodiment, the concentration of the marker is determined in a body sample taken before treatment of an intervertebral disc defect, in particular prior to a sequestrectomy, and in a body sample taken after the treatment of the intervertebral disc defect. Preferably, a test is conducted to determine whether the concentration of the marker in the body sample taken after the treatment of the intervertebral disc defect is increased or decreased compared to the concentration of the marker in the body sample taken before the treatment of the intervertebral disc defect, in particular before the sequestrectomy.

Preferably, an elevated or decreased concentration of the marker is correlated with the prediction of a positive clinical outcome of an autologous disc cell transplantation, in particular a matrix-associated autologous disc cell transplantation, and/or with a positive progress assessment/progress monitoring of an autologous disc cell transplantation, in particular a matrix-associated autologous disc cell transplantation, and/or with a positive assessment of the quality of an implant and/or advanced therapy medicinal products (ATMP) for the treatment of an intervertebral disc defect and/or for the reconstruction of intervertebral disc tissue, or is correlated with the prediction of a negative clinical outcome of an autologous disc cell transplantation, in particular a matrix-associated autologous disc cell transplantation, and/or with a negative progress assessment/progress monitoring of an autologous disc cell transplantation, in particular a matrix-associated autologous disc cell transplantation, and/or with a negative assessment of the quality of an implant and/or advanced therapy medicinal products (ATMP) for the treatment of an intervertebral disc defect and/or for the reconstruction of intervertebral disc tissue.

In a further embodiment, the body samples are serum samples, plasma samples, urine samples, intervertebral-disc-cell-containing samples, or a combination of two or more of said body sample types. Accordingly, in a further embodiment, the at least one body sample mentioned in the above paragraphs can be at least one serum sample, at least one plasma sample, at least one urine sample, at least one intervertebral-disc-cell-containing sample, or a combination of two or more of said body sample types.

In a further embodiment, the concentration of CTX-I is determined in body samples taken from a patient at different times. A decrease in the concentration of CTX-I over time, i.e. a decreasing concentration of CTX-I as the body samples are taken, is preferably tested. The body samples are preferably serum samples.

Preferably, a decrease in the concentration of CTX-I over time is correlated with the prediction of a positive clinical outcome of an autologous disc cell transplantation, in particular a matrix-associated autologous disc cell transplantation, and/or with a positive progress assessment/progress monitoring of an autologous disc cell transplantation, in particular a matrix-associated autologous disc cell transplantation, and/or with a positive assessment of the quality of an implant and/or advanced therapy medicinal products (ATMP) for the treatment of an intervertebral disc defect and/or for the reconstruction of intervertebral disc tissue. In particular, a decrease in the concentration of CTX-I over time can be correlated with reduced breakdown of collagen type I or reduced breakdown of intervertebral disc tissue.

In a further embodiment, the concentration of CTX-I is determined in a body sample taken before treatment of an intervertebral disc defect, in particular prior to a sequestrectomy, and in a body sample taken after the treatment of the intervertebral disc defect. Preferably, a test is conducted to determine whether the concentration of CTX-I in the body sample taken after the treatment of the intervertebral disc defect is decreased compared to the concentration of CTX-I in the body sample taken before the treatment of the intervertebral disc defect, in particular before the sequestrectomy. The body samples are preferably serum samples.

Preferably, a decreased concentration of CTX-I is correlated with the prediction of a positive clinical outcome of an autologous disc cell transplantation, in particular a matrix-associated autologous disc cell transplantation, and/or with a positive progress assessment/progress monitoring of an autologous disc cell transplantation, in particular a matrix-associated autologous disc cell transplantation, and/or with a positive assessment of the quality of an implant and/or advanced therapy medicinal products (ATMP) for the treatment of an intervertebral disc defect and/or for the reconstruction of intervertebral disc tissue. In particular, a decreased concentration of CTX-I can be correlated with reduced breakdown of collagen type I or reduced breakdown of intervertebral disc tissue.

In a further embodiment, the concentration of CTX-II is determined in body samples taken from a patient at different times. Preferably, a decrease in the concentration of CTX-II over time, i.e. a decreasing concentration of CTX-II as the body samples are taken, is tested. The body samples are preferably serum samples.

Preferably, a decrease in the concentration of CTX-II over time is correlated with the prediction of a positive clinical outcome of an autologous disc cell transplantation, in particular a matrix-associated autologous disc cell transplantation, and/or with a positive progress assessment/progress monitoring of an autologous disc cell transplantation, in particular a matrix-associated autologous disc cell transplantation, and/or with a positive assessment of the quality of an implant and/or advanced therapy medicinal products (ATMP) for the treatment of an intervertebral disc defect and/or for the reconstruction of intervertebral disc tissue.

In a further embodiment, the concentration of CTX-II is determined in a body sample taken before treatment of an intervertebral disc defect, in particular prior to a sequestrectomy, and in a body sample taken after the treatment of the intervertebral disc defect. Preferably, a test is conducted to determine whether the concentration of CTX-II in the body sample taken after the treatment of the intervertebral disc defect is decreased compared to the concentration of CTX-II in the body sample taken before the treatment of the intervertebral disc defect, in particular before the sequestrectomy. The body samples are preferably serum samples.

Preferably, a decreased concentration of CTX-II is correlated with the prediction of a positive clinical outcome of an autologous disc cell transplantation, in particular a matrix-associated autologous disc cell transplantation, and/or with a positive progress assessment/progress monitoring of an autologous disc cell transplantation, in particular a matrix-associated autologous disc cell transplantation, and/or with a positive assessment of the quality of an implant and/or advanced therapy medicinal products (ATMP) for the treatment of an intervertebral disc defect and/or for the reconstruction of intervertebral disc tissue.

In a further embodiment, the concentration of NTX-I is determined in body samples taken from a patient at different times. Preferably, a decrease in the concentration of NTX-I over time, i.e. a decreasing concentration of NTX-I as the body samples are taken, is tested. The body samples are preferably serum and/or urine samples.

Preferably, a decrease in the concentration of NTX-I over time is correlated with the prediction of a positive clinical outcome of an autologous disc cell transplantation, in particular a matrix-associated autologous disc cell transplantation, and/or with a positive progress assessment/progress monitoring of an autologous disc cell transplantation, in particular a matrix-associated autologous disc cell transplantation, and/or with a positive assessment of the quality of an implant and/or advanced therapy medicinal products (ATMP) for the treatment of an intervertebral disc defect and/or for the reconstruction of intervertebral disc tissue. In particular, a decrease in the concentration of NTX-I over time can be correlated with a decreasing or reduced breakdown of collagen type I in a treated intervertebral disc.

In a further embodiment, the concentration of NTX-I is determined in a body sample taken before treatment of an intervertebral disc defect, in particular before a sequestrectomy, and in a body sample taken after the treatment of the intervertebral disc defect. Preferably, a test is conducted to determine whether the concentration of NTX-I in the body sample taken after the treatment of the intervertebral disc defect is decreased compared to the concentration of NTX-I in the body sample taken before the treatment of the intervertebral disc defect, in particular before the sequestrectomy. The body samples are preferably serum and/or urine samples.

Preferably, a decreased concentration of NTX-I is correlated with the prediction of a positive clinical outcome of an autologous disc cell transplantation, in particular a matrix-associated autologous disc cell transplantation, and/or with a positive progress assessment/progress monitoring of an autologous disc cell transplantation, in particular a matrix-associated autologous disc cell transplantation, and/or with a positive assessment of the quality of an implant and/or advanced therapy medicinal products (ATMP) for the treatment of an intervertebral disc defect and/or for the reconstruction of intervertebral disc tissue. In particular, a decreased concentration of NTX-I can be correlated with a decreasing or reduced breakdown of collagen type I in a treated intervertebral disc.

In a further embodiment, the concentration of CPII is determined in body samples taken from a patient at different times. Preferably, an increase in the concentration of CPII over time, i.e. an increase in the concentration of CPII as the body samples are taken, is tested. The body samples are preferably serum samples.

Preferably, an increase in the concentration of CPII over time is correlated with the prediction of a positive clinical outcome of an autologous disc cell transplantation, in particular a matrix-associated autologous disc cell transplantation, and/or with a positive progress assessment/progress monitoring of an autologous disc cell transplantation, in particular a matrix-associated autologous disc cell transplantation, and/or with a positive assessment of the quality of an implant and/or advanced therapy medicinal products (ATMP) for the treatment of an intervertebral disc defect and/or for the reconstruction of intervertebral disc tissue. In particular, an increase in the concentration of CPII over time can be correlated with enhanced synthesis activity of collagen type II and/or enhanced cartilage tissue synthesis activity of transplanted intervertebral disc cells.

In a further embodiment, the concentration of CPII is determined in a body sample taken before treatment of an intervertebral disc defect, in particular before a sequestrectomy, and in a body sample taken after the treatment of the intervertebral disc defect. Preferably, a test is conducted to determine whether the concentration of CPII in the body sample taken after the treatment of the intervertebral disc defect is increased compared to the concentration of CPII in the body sample taken before the treatment of the intervertebral disc defect, in particular before the sequestrectomy. The body samples are preferably serum samples.

Preferably, an elevated concentration of CPII is correlated with the prediction of a positive clinical outcome of an autologous disc cell transplantation, in particular a matrix-associated autologous disc cell transplantation, and/or with a positive progress assessment/progress monitoring of an autologous disc cell transplantation, in particular a matrix-associated autologous disc cell transplantation, and/or with a positive assessment of the quality of an implant and/or advanced therapy medicinal products (ATMP) for the treatment of an intervertebral disc defect and/or for the reconstruction of intervertebral disc tissue. In particular, an elevated concentration of CPII can be correlated with enhanced synthesis activity of collagen type II and/or enhanced cartilage tissue synthesis activity of transplanted intervertebral disc cells.

In a further embodiment, the concentration of COMP is determined in body samples taken from a patient at different times. Preferably, a decrease in the concentration of COMP over time, i.e. a decreasing concentration of COMP as the body samples are taken, is tested. The body samples are preferably serum samples.

Preferably, a decrease in the concentration of COMP over time is correlated with the prediction of a positive clinical outcome of an autologous disc cell transplantation, in particular a matrix-associated autologous disc cell transplantation, and/or with a positive progress assessment/progress monitoring of an autologous disc cell transplantation, in particular a matrix-associated autologous disc cell transplantation, and/or with a positive assessment of the quality of an implant and/or advanced therapy medicinal products (ATMP) for the treatment of an intervertebral disc defect and/or for the reconstruction of intervertebral disc tissue. In particular, a decrease in the concentration of COMP over time can be correlated with reduced cartilage tissue breakdown in a treated intervertebral disc and/or with a lesser decrease in the volume or the water content of a treated intervertebral disc.

In a further embodiment, the concentration of COMP is determined in a body sample taken before treatment of an intervertebral disc defect, in particular before a sequestrectomy, and in a body sample taken after the treatment of the intervertebral disc defect. Preferably, a test is conducted to determine whether the concentration of COMP in the body sample taken after the treatment of the intervertebral disc defect is decreased compared to the concentration of COMP in the body sample taken before the treatment of the intervertebral disc defect, in particular before the sequestrectomy. The body samples are preferably serum samples.

Preferably, a decreased concentration of COMP is correlated with the prediction of a positive clinical outcome of an autologous disc cell transplantation, in particular a matrix-associated autologous disc cell transplantation, and/or with a positive progress assessment/progress monitoring of an autologous disc cell transplantation, in particular a matrix-associated autologous disc cell transplantation, and/or with a positive assessment of the quality of an implant and/or advanced therapy medicinal products (ATMP) for the treatment of an intervertebral disc defect and/or for the reconstruction of intervertebral disc tissue. In particular, a decreased concentration of COMP can be correlated with reduced cartilage tissue breakdown in a treated intervertebral disc and/or with a lesser decrease in the volume or the water content of a treated intervertebral disc.

In a further embodiment, the concentration of TNF-α is determined in body samples taken from a patient at different times. Preferably, a decrease in the concentration of TNF-α over time, i.e. a decreasing concentration of TNF-α as the body samples are taken, is tested. The body samples are preferably serum samples.

Preferably, a decrease in the concentration of TNF-α over time is correlated with the prediction of a positive clinical outcome of an autologous disc cell transplantation, in particular a matrix-associated autologous disc cell transplantation, and/or with a positive progress assessment/progress monitoring of an autologous disc cell transplantation, in particular a matrix-associated autologous disc cell transplantation, and/or with a positive assessment of the quality of an implant and/or advanced therapy medicinal products (ATMP) for the treatment of an intervertebral disc defect and/or for the reconstruction of intervertebral disc tissue. In particular, a decrease in the concentration of TNF-α over time can be correlated with a decrease in inflammation/degeneration of a treated intervertebral disc.

In a further embodiment, the concentration of TNF-α is determined in a body sample taken before treatment of an intervertebral disc defect, in particular before a sequestrectomy and in a body sample taken after the treatment of the intervertebral disc defect. Preferably, a test is conducted to determine whether the concentration of TNF-α in the body sample taken after the treatment of the intervertebral disc defect is decreased compared to the concentration of TNF-α in the body sample taken before the treatment of the intervertebral disc defect, in particular before the sequestrectomy. The body samples are preferably serum samples.

Preferably, a decreased concentration of TNF-α is correlated with the prediction of a positive clinical outcome of an autologous disc cell transplantation, in particular a matrix-associated autologous disc cell transplantation, and/or with a positive progress assessment/progress monitoring of an autologous disc cell transplantation, in particular a matrix-associated autologous disc cell transplantation, and/or with a positive assessment of the quality of an implant and/or advanced therapy medicinal products (ATMP) for the treatment of an intervertebral disc defect and/or for the reconstruction of intervertebral disc tissue. In particular, a decreased concentration of TNF-α can be correlated with a decrease in inflammation/degeneration of a treated intervertebral disc.

In a further embodiment, the concentration of IL-4 is determined in body samples taken from a patient at different times. Preferably, a decrease in the concentration of IL-4 over time, i.e. a decreasing concentration of IL-4 as the body samples are taken, is tested. The body samples are preferably serum samples.

Preferably, a decrease in the concentration of IL-4 over time is correlated with the prediction of a positive clinical outcome of an autologous disc cell transplantation, in particular a matrix-associated autologous disc cell transplantation, and/or with a positive progress assessment/progress monitoring of an autologous disc cell transplantation, in particular a matrix-associated autologous disc cell transplantation, and/or with a positive assessment of the quality of an implant and/or advanced therapy medicinal products (ATMP) for the treatment of an intervertebral disc defect and/or for the reconstruction of intervertebral disc tissue. In particular, a decrease in the concentration of IL-4 over time can be correlated with the decrease in inflammation/degeneration of a treated intervertebral disc.

In a further embodiment, the concentration of IL-4 is determined in a body sample taken before treatment of an intervertebral disc defect, in particular before a sequestrectomy, and in a body sample taken after the treatment of the intervertebral disc defect. Preferably, a test is conducted to determine whether the concentration of IL-4 in the body sample taken after the treatment of the intervertebral disc defect is decreased compared to the concentration of IL-4 in the body sample taken before the treatment of the intervertebral disc defect, in particular before the sequestrectomy. The body samples are preferably serum samples.

Preferably, a decreased concentration of IL-4 is correlated with the prediction of a positive clinical outcome of an autologous disc cell transplantation, in particular a matrix-associated autologous disc cell transplantation, and/or with a positive progress assessment/progress monitoring of an autologous disc cell transplantation, in particular a matrix-associated autologous disc cell transplantation, and/or with a positive assessment of the quality of an implant and/or advanced therapy medicinal products (ATMP) for the treatment of an intervertebral disc defect and/or for the reconstruction of intervertebral disc tissue. In particular, a decreased concentration of IL-4 can be correlated with the decrease in inflammation/degeneration of a treated intervertebral disc.

In a further embodiment, a body sample, preferably a body sample taken before an autologous disc cell transplantation, matrix-associated autologous disc cell transplantation, implant-based intervertebral disc treatment, or advanced therapy medicinal product-based intervertebral disc treatment, is tested for a decreased concentration of IL-1ra. The body sample is preferably a serum sample.

Preferably, a decreased concentration of IL-1ra is correlated with the presence of an intervertebral disc defect, in particular an intervertebral disc extrusion. In other words, a decreased concentration of IL-1ra is preferably used as a marker for the presence of an intervertebral disc defect, in particular an intervertebral disc extrusion.

In a further embodiment, a body sample, preferably a body sample taken before an autologous disc cell transplantation, matrix-associated autologous disc cell transplantation, implant-based intervertebral disc treatment, or advanced therapy medicinal product-based intervertebral disc treatment, is tested for a decreased concentration of CTX-II. The body sample is preferably a serum sample.

Preferably, a decreased concentration of CTX-II is correlated with the presence of an intervertebral disc defect, in particular an intervertebral disc extrusion.

In a further embodiment, the concentration of the marker in the supernatant of a cell culture containing autologous intervertebral disc cells is tested.

The intervertebral disc cells are preferably intervertebral disc cells taken from tissue obtained from a removed intervertebral slipped disc. In other words, the intervertebral disc cells are preferably cells removed from the patient during a sequestrectomy or nucleotomy.

In a further embodiment, the cell culture supernatant is tested for an elevated concentration of VEGF. Preferably, an elevated concentration of VEGF is correlated with the presence of an intervertebral disc defect, in particular an intervertebral disc extrusion, and/or spinal column defect and/or with a negative assessment of the quality of the intervertebral disc cells, preferably with respect to their usability for an autologous disc cell transplantation, in particular a matrix-associated autologous disc cell transplantation. The term “elevated concentration” used in this paragraph means that the concentration of VEGF is increased compared to the concentration of VEGF in the supernatant of a cell culture comprising intervertebral disc cells from a patient without an intervertebral disc defect, in particular without an intervertebral disc extrusion and/or without a vertebral defect.

In a further embodiment, the cell culture supernatant is tested for a decreased concentration of MMP-3. Preferably, a decreased concentration of MMP-3 is correlated with the presence of an intervertebral disc defect, in particular an intervertebral disc extrusion, and/or a spinal column defect and/or with a negative assessment of the quality of the intervertebral disc cells, preferably with respect to their usability for an autologous disc cell transplantation, in particular a matrix-associated autologous disc cell transplantation. The term “decreased concentration” used in this paragraph means that the concentration of MMP-3 is decreased compared to the concentration of MMP-3 in the supernatant of a cell culture comprising intervertebral disc cells from a patient without an intervertebral disc defect, in particular without an intervertebral disc extrusion and/or without a vertebral defect.

In a further embodiment, the concentration of the marker or markers is tested by means of Western blot, protein chips, antibodies, immunoassays such as ELISA (enzyme-linked immunosorbent assay) or LUMINEX immunoassays or immunohistochemical methods.

The invention further relates to an in vitro method for the

prediction (prognosis) of a clinical outcome (so-called outcome prognosis) of an autologous disc cell transplantation, in particular a matrix-associated autologous disc cell transplantation,
and/or

progress assessment/progress monitoring of an autologous disc cell transplantation, in particular a matrix-associated autologous disc cell transplantation,
and/or

assessment of the quality of intervertebral disc cells, i.e. so-called intervertebral disc chondrocytes, preferably for an autologous disc cell transplantation, in particular for a matrix-associated autologous disc cell transplantation,
and/or

assessment of the quality of an implant and/or advanced therapy medicinal products (ATMP) for the treatment of an intervertebral disc defect and/or for the reconstruction of intervertebral disc tissue
and/or

diagnosis of an intervertebral disc defect, in particular a lumbar, preferably lumbar-sacral, intervertebral disc defect, and/or spinal column defect, wherein a marker is used that is selected from the group consisting of CTX-I, CTX-II, NTX-I, CPII, VEGF, C2C, MMP-3, hyaluronic acid, TNF-α, IL-1ra, IL-4, COMP, YKL-40 and combinations of at least two of said markers.

In order to avoid repetition, with respect to further features and advantages of the method, in particular of the marker or of the markers, reference is made to the explanations made in the context of the above description in their entirety. The embodiments described therein also apply to the method according to the invention.

Moreover, the invention relates to the use of a kit for carrying out a method according to the invention. The kit comprises at least one substance/at least one agent for determining the concentration of a marker selected from the group consisting of CTX-I, CTX-II, NTX-I, CPII, VEGF, C2C, MMP-3, hyaluronic acid, TNF-α, IL-1ra, IL-4, COMP, YKL-40 and combinations of at least two of said markers.

The at least one substance or the at least one agent can in particular be selected from the group consisting of antibodies such as capture and/or detection antibodies, fluorescent dye, beads, buffer solution, nutrient solution, washing solution and combinations of two or more of said substances or agents.

In order to avoid repetition, with respect to further features and advantages of the kit, in particular of the marker or of the markers, reference is also made to the explanations made in the context of the above description in their entirety. The embodiments described therein also apply, whenever possible, to use of the kit according to the invention.

Finally, the invention relates to the use in vitro of a marker for the

prediction (prognosis) of a clinical outcome (so-called outcome prognosis) of an autologous disc cell transplantation, in particular a matrix-associated autologous disc cell transplantation,
and/or

progress assessment/progress monitoring of an autologous disc cell transplantation, in particular a matrix-associated autologous disc cell transplantation,
and/or

assessment of the quality of intervertebral disc cells, i.e. so-called intervertebral disc chondrocytes, preferably for an autologous disc cell transplantation, in particular for a matrix-associated autologous disc cell transplantation,
and/or

assessment of the quality of an implant and/or advanced therapy medicinal products (ATMP) for the treatment of an intervertebral disc defect and/or for the reconstruction of intervertebral disc tissue
and/or

diagnosis of an intervertebral disc defect, in particular a lumbar, preferably lumbar-sacral, intervertebral disc defect, and/or spinal column defect, wherein the marker is selected from the group consisting of CTX-I, CTX-II, NTX-I, CPII, VEGF, C2C, MMP-3, hyaluronic acid, TNF-α, IL-1ra, IL-4, COMP, YKL-40 and combinations of at least two of said markers.

In order to avoid repetition, with respect to further features and advantages of use, in particular of the marker or of the markers, reference is also made to the explanations made in the context of the above description in their entirety. The embodiments described therein also apply to this use according to the invention.

Further features and advantages of the invention can be found in the following description of preferred embodiments in the form of examples. In this context, individual features can be implemented individually or in combination with one another. The examples described below serve solely to further explain the invention, and the invention is not limited to said examples.

### EXAMPLE SECTION

### 1. Methods

1.1 Implants

In a phase I study, 20 patients who had suffered a slipped intervertebral disc were treated either with an implant referred to as the NOVOCART® Disc basic (9 patients) or an implant referred to as the NOVOCART® Disc plus (11 patients). Both the NOVOCART® Disc basic and the NOVOCART® Disc plus are intended for regeneration of the nucleus pulposus and/or annulus fibrosus.

The implant NOVOCART® Disc basic is composed of two components, namely a first component, which comprises maleimide-functionalized albumin (“modified maleimido-albumin”) and hyaluronic acid, and a second component, which comprises the crosslinking agent bis-thio-polyethylene glycol. The first component and the second component are each in the form of an aqueous solution.

The implant NOVOCART Disc plus can be obtained based on NOVOCART® Disc basic by adding autologous intervertebral disc cells to the first component. Accordingly, the implant NOVOCART Disc plus was composed of the following two components: a first component, which comprises maleimide-functionalized albumin, hyaluronic acid, autologous intervertebral disc cells, and a transplantation or cell culture medium (solution with glucose and supplements such as amino acids and vitamins), and a second component, which comprises bis-thio-polyethylene glycol as a crosslinking agent. The first component of NOVOCART® Disc plus was in the form of an aqueous suspension, while the second component was in the form of an aqueous solution. According to EC Regulation No. 1394/2007 on advanced therapy medicinal products, the product constituted a tissue-engineering product.

1.2 Obtaining of Autologous Intervertebral Disc Cells

The autologous intervertebral disc cells were obtained from the tissue removed in the sequestrectomy. The tissue was mechanically reduced in size and then subjected to collagenase/protease digestion. In order to exclude mixed cultures, the cell suspension obtained was kept in suspension for a further 48 hours. This was followed by expansion of the isolated intervertebral disc cells for a further 12 days (±2). The cells were cryopreserved until transplantation. At a specified time before transplantation, the cells were thawed and again expanded for several days.

In order to produce the implant NOVOCART® Disc plus, the cells were mixed after harvesting with an aqueous solution comprising maleimide-functionalized albumin and hyaluronic acid in the above-mentioned transplantation medium.

The obtaining, isolation, cultivation, cellular and molecular biological characterization, quality testing, and sterile control of the autologous intervertebral disc cells were carried out under GMP conditions.

The first component of NOVOCART Disc plus contained approx. 4 million human intervertebral disc cells/ml (±10%) in 2.65 ml of medium, 0.02 g of hyaluronic acid, and 0.35 ml of maleimide-functionalized albumin (total of 4 ml). The second component of NOVOCART® Disc plus contained 1 ml of bis-thio-polyethylene glycol as a crosslinking agent.

1.3 Supernatant of the Patient Cell Culture

The supernatants of the patient cell cultures were collected in harvesting cells for the production of NOVOCART® Disc plus. These cell cultures were also prepared for the patients treated with NOVOCART Disc basic. For each cell culture supernatant, a corresponding blank medium (containing the same human serum) was measured concurrently, and the measurement values of the samples were corrected therewith (blank). This was done in order to take into account the fact that different serum loads were used for the various media used in cell cultivation. The measurement values of the cell culture supernatants were also standardized to the corresponding cell count at the time of the harvest and the residence time of the medium on the cells (concentration of the analyte/h/mio. cells). The results obtained in this manner therefore reflected the synthesis capacity of the cells.

The following analytes were determined in the cell culture supernatants: Bone alkaline phosphatase (BAP), bone morphogenetic protein-2 (BMP-2), cathepsin K, cartilage oligomeric protein (COMP), aggrecan epitope CS 846, hyaluronic acid (HA), matrix metalloproteinase-3 (MMP-3), transforming growth factor-β1 (TGF-β1), transforming growth factor-β2 (TG-β2), transforming growth factor-β3 (TG-β3), cartilage glycoprotein-39 (YKL-40), interleukin-1β (IL-1β), interleukin-4 (IL-4), interleukin-6 (IL-6), interleukin-8 (IL-8), interleukin-10 (IL-10), interleukin-1 receptor antagonist (IL-1ra), vascular endothelial growth factor (VEGF), interferon-y (IFN-y), tumor necrosis factor-α (TNF-α), RANTES (regulated on activation, normal T cell expressed and secreted) and CCL5 (chemokine (C-C motif) ligand 5).

1.4 Blood and Urine Samples

Blood and urine samples of the patients treated with NOVOCART Disc basic or NOVOCART Disc plus were collected at the following times:

1. on the day of sequestrectomy (day 0)
2. 90±15 days (ReOP implantation) after sequestrectomy
3. 42±7 days (visit 4) after transplantation
4. 90±7 days (visit 5) after transplantation
5. 180±14 days (visit 6) after transplantation
6. 365±14 days (visit 7) after transplantation

After receipt at the clinical centers, the samples were sent to the Applicant. Urine samples were directly divided into aliquot parts and stored at −20° C. until the time of testing. Serum was obtained from the blood samples by centrifugation at 1500 rpm for 15 min, and this serum was then also divided into aliquot parts and stored at −20° C. until the time of testing.

The following analytes were determined in the serum samples: Collagen type II, neoepitope C2C (C2C), COMP, C-terminal peptide of collagen type II procollagen (CPII), N-terminal peptide of collagen type II procollagen (PIIANP), CS 846, C-terminal telopeptide of collagen type I (CTX-I), C-terminal telopeptide of collagen type II (CTX-II), N-terminal telopeptide of collagen type I (NTX-I), hyaluronic acid, YKL-40, interleukin-1β (IL-1β), interleukin-4 (IL-4), interleukin-6 (IL-6), interleukin-8 (IL-8), interleukin-10 (IL-10), interleukin-1 receptor antagonist (IL-1ra), vascular endothelial growth factor (VEGF), interferon-y (IFN-y), tumor necrosis factor-α (TNF-α), RANTES (regulated on activation, normal T cell expressed and secreted), and CCL5 (chemokine (C-C motif) ligand 5).

The following analytes were determined in the urine samples:

C2C, CTX-I, NTX-I, CTX-II. The measurement values of the four analytes were standardized to the creatinine contained in the urine (concentration of analyte/mM of creatinine).

1.5 MRT Imaging:

On the days of the ReOP and of visits 4 to 7, the patients were also examined by MRT. The MRT images were evaluated by an outside expert according to the following criteria:

volume (in mm³) of the central intervertebral disc (treated intervertebral disc) and the intervertebral disc caudal or cranial thereto

Modic score of the central, caudal and cranial intervertebral disc

Pfirrman score of the central, caudal and cranial intervertebral disc

Dorsal extrusion of the central, caudal and cranial intervertebral disc

T2 relaxation time (in ms) of the central, caudal and cranial intervertebral disc.

1.6 Measurement of the Markers:

Measurement of the analytes was carried out by means of commercially available kits. In this case, planar ELISAs (enzyme-linked immunosorbent assays) in the microtiter plate format and a bead-based multiplex method (Luminex technology) were used. In the planar ELISAs or immunoassays, both classical assays and sandwich assays, as well as competitive assays, were used. Measurement was conducted according to the indications of the respective manufacturer.

1.7 Statistical Evaluation:

Version 2.5 of the statistics program SigmaStat (Systat, USA) and Microsoft Excel 2011 (Microsoft Corporation, USA) were used for data analysis. In evaluation of the measurement data in Excel, descriptive methods such as mean value, standard deviation, and standard error were used. The data were analyzed using the normal distribution (Kolmogorov-Smirnov test). Student's t test was used in comparison of two groups with normally distributed measurement values, and a non-parametric test (Mann-Whitney) was conducted for the values not showing normal distribution. For comparison of more than two groups with one another, ANOVA and a suitable post hoc test were used.

Correlations were calculated using Spearman's rank correlation (for data that were not normally distributed) or the Pearson product moment correlation (for normally distributed data). A p value of <0.05 for differences and correlations was considered to be statistically significant.

The results of the marker measurements were evaluated with respect to the following aspects:

Are there significant changes in the serum or urine concentration of the patients over the course of the study based on day 0 (baseline)?

Are there significant differences between the two groups treated with NOVOCART® Disc basic and NOVOCART® Disc plus respectively?

Do marker concentrations correlate with the radiological findings (MRT evaluations)?

### 2. Results of the Study

2.1 Significant Changes in Biomarkers in the Study Over the Course of Time

The biomarkers that showed significant changes during the observation period are listed in the following. In all cases, these significant changes are based on the measurement values on day 0 (the day of sequestrectomy), i.e. the baseline value. The pooled data, i.e. the data both for the patients treated with the NOVOCART® Disc basic and the patients treated with the NOVOCART® Disc plus, are taken into account.

2.1.1 CTX-I (Alpha-CTX, Degradation Marker)

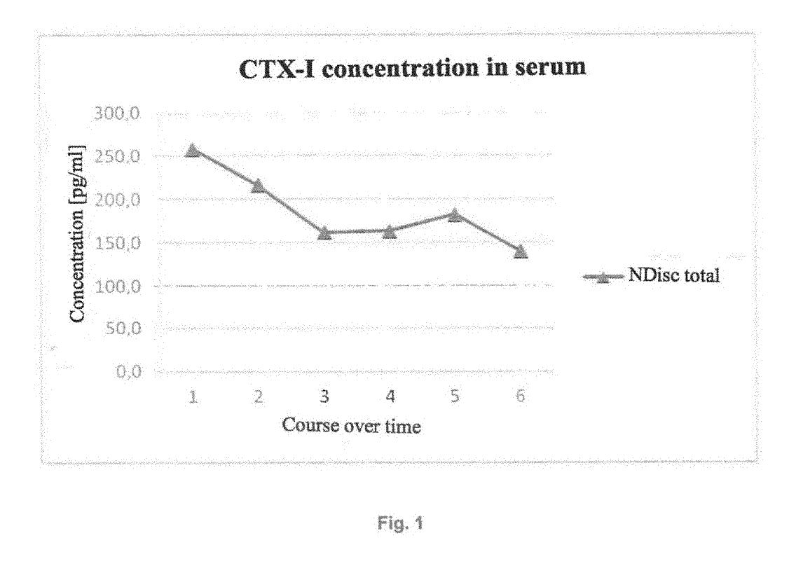

The concentration of CTX-I in the serum of the patients decreased during the observation period. There was a significant difference between day 0 (i.e. pre-treatment) and visits 4 (1.5 months after treatment) and 7 (12 months after treatment). The mean values for all of the treated patients were 259 pg/ml on day 0, 171 pg/ml on visit 4, and 140 pg/ml on visit 7. These mean values are given below together with the standard deviations in Table 1. The course over time of the concentration of CTX-I in the serum is shown for all of the patients in FIG. 1.

TABLE 1

| | Day 0 | ReOP | V4 | V5 | V6 | V7 |
|---|---|---|---|---|---|---|
| Mean | 258.7 | 216.7 | 162.4 | 163.9 | 182.6 | 140.1 |
| SD | 113.2 | 136.8 | 66.3 | 66.7 | 82.5 | 53.2 |

2.1.2 CTX-II (Degradation Markers)

The concentration of CTX-II in the urine of the patients decreased during the observation period. There was a significant difference between day 0 (i.e. pre-treatment) and visit 5 (3 months after treatment), visit 6 (6 months after treatment) and visit 7 (12 months after treatment).

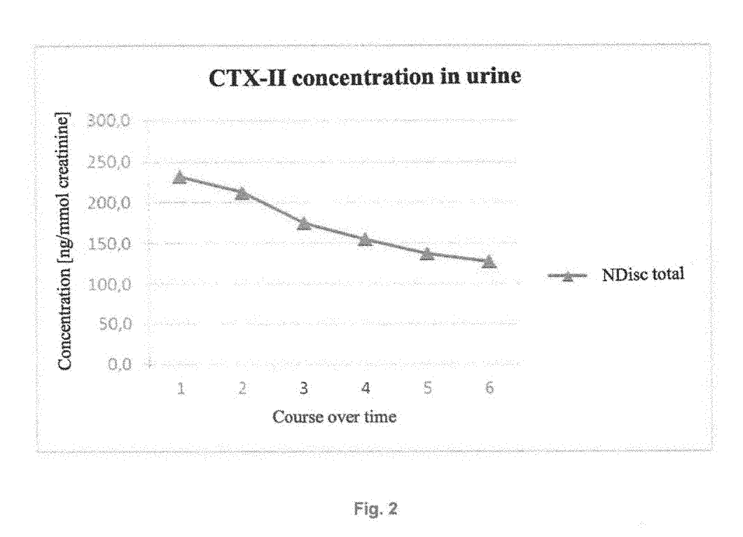

The mean values for all of the treated patients were 233 ng/mmol creatinine on day 0, 156 ng/mmol creatinine on visit 5, 138 ng/mmol creatinine on visit 6, and 128 pg/ml on visit 7. The mean values and the corresponding standard deviations are given below in Table 2. The course over time of the concentration of CTX-II in the urine is given for all of the patients in FIG. 2.

TABLE 2

| | Day 0 | ReOP | V4 | V5 | V6 | V7 |
|---|---|---|---|---|---|---|
| Mean | 232.5 | 213.1 | 175.7 | 155.7 | 137.7 | 127.8 |
| SD | 96.4 | 94.4 | 90.2 | 71.8 | 54.9 | 55.4 |

2.1.3 NTX-I (Degradation Markers)

The concentration of NTX-I in the serum of the patients decreased during the observation period. There was a significant difference between day 0 (i.e. pre-treatment) and visit 5 (3 months after treatment), visit 6 (6 months after treatment) and visit 7 (12 months after treatment).

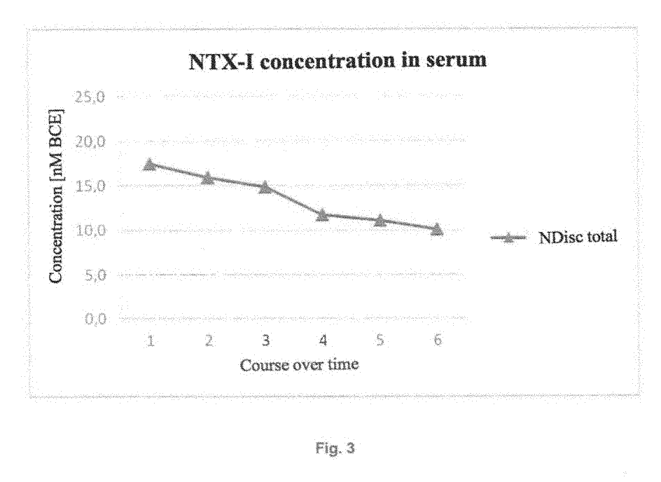

The mean values for all of the treated patients were 17 nM BCE on day 0, 12 nM BCE on visit 5, 11 nM BCE on visit 6, and 10 nM BCE on visit 7. The mean values and the corresponding standard deviations are given below in Table 3. The course over time of the concentration of NTX-I in the serum is given for all of the treated patients in FIG. 3.

TABLE 3

| | Day 0 | ReOP | V4 | V5 | V6 | V7 |
|---|---|---|---|---|---|---|
| Mean | 17.5 | 15.9 | 14.9 | 11.7 | 11.1 | 10.1 |
| SD | 5.4 | 5.3 | 3.5 | 5.8 | 3.6 | 2.9 |

In line with the serum concentrations, the concentration of NTX-I in the urine of the patients also decreased. There was a significant difference between day 0 (i.e. pre-treatment) and visit 4 (1.5 months after treatment), visit 5 (3 months after treatment) and visit 7 (12 months after treatment).

The mean values for all of the treated patients were 37 nM/mM creatinine on day 0, 25 nM/mM creatinine on visit 4, 28 nM/mM on visit 5, and 23.5 nM/mM creatinine on visit 7.

2.1.4 CPII (PIICP, Chondrocalcin, Formation Marker)

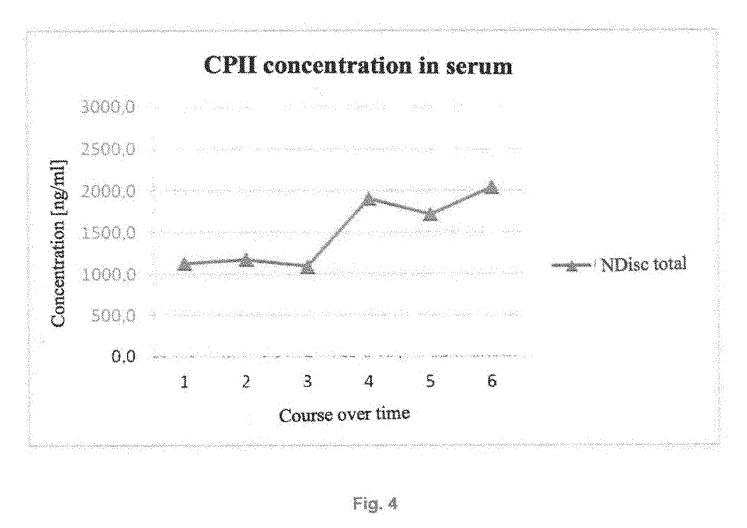

The concentrations of CPII in the serum of the patients increased during the observation period. There was a significant difference between day 0 (i.e. pre-treatment) and visits 5 (3 months after treatment) and 7 (12 months after treatment). The mean values for all of the treated patients were 1133 ng/ml on day 0, 1916 ng/ml on visit 5, and 2045 ng/ml on visit 7. The mean values and the corresponding standard deviations are given below in Table 4. The course over time of the concentration of CPII in the serum is given for all of the treated patients in FIG. 4.

TABLE 4

| | Day 0 | ReOP | V4 | V5 | V6 | V7 |
|---|---|---|---|---|---|---|
| Mean | 1133.0 | 1175.2 | 1091.2 | 1916.0 | 1722.5 | 2044.9 |
| SD | 548.4 | 539.9 | 406.2 | 672.3 | 708.1 | 1086.4 |

### Conclusion

A significant decrease was confirmed for typical collagen degradation markers during the observation period. At the same time, an increase was observed for the collagen type II synthesis marker CPII. Taken together, these results indicate a positive course of healing and/or an improvement in the health status of the patients. Moreover, a decrease in the concentration of CTX-II is also to be evaluated as positive, as an increased serum level is generally accompanied by a disease progression.

In addition to the above-mentioned markers, COMP also varied in the early measurements (between day 0 and ReOP and day 0 and visit 4). There were also significant differences over time in the concentration of VEGF. Significant differences were observed between day 0, ReOP and visit 4 and visits 5 and 7 respectively.

2.2 Significant Changes in Biomarkers Over Time on Comparison of the Patient Groups with One Another

2.2.1 CPII (PIICP, Chondrocalcin, Formation Marker)

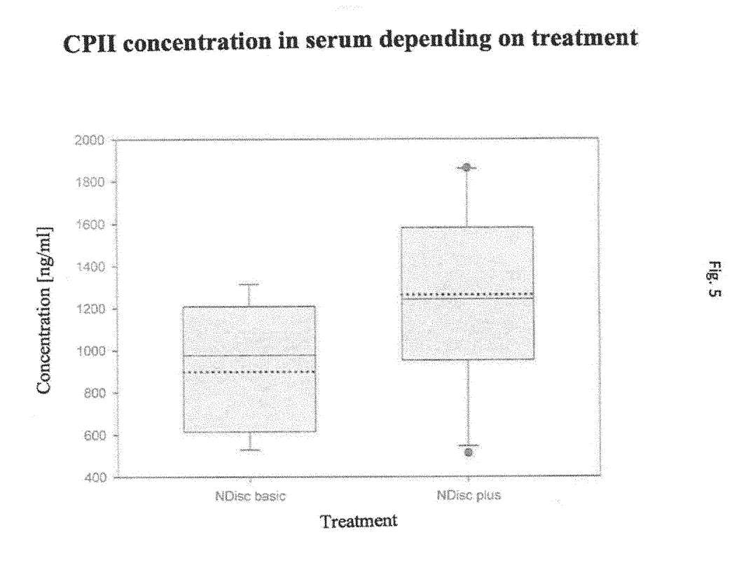

The serum concentrations of CPII in the patient group treated with NOVOCART Disc basic were significantly lower at the time of visit 4 (i.e. 3 months after treatment) than in the patient group treated with NOVOCART Disc plus. The differences in the concentrations of CPII are shown in FIG. 5.

### Conclusion

For CPII, the serum concentration was lower in the cell-free group treated with NOVOCART® Disc basic. This indicates that in the patient group treated with NOVOCART® Disc basic, there was less intervertebral disc tissue turnover than in the patient group treated with NOVOCART® Disc plus.

2.3 Correlation of Biomarkers with the Radiological Finding of Extrusion

In this case, MRT findings at the time of the ReOP and visit 4 (1.5 months after treatment) were correlated with the biomarker concentrations.

Result:

At the time of the ReOP, there were significant differences or a significant correlation of the radiological finding “presence of extrusion” (central intervertebral disc) with the concentration of interleukin-1 receptor antagonists (IL1ra) in the serum. The patients who showed an extrusion on MRT had less IL1ra in the serum (2.3 ng/ml) than patients without signs of an extrusion (10.8 ng/ml).

Significant differences were also found for CTX-II.

A significant difference between the patients with radiologically established extrusion and those without and a significant correlation between the concentrations and the occurrence of an extrusion were seen in VEGF in the cell culture supernatants. Cell cultures of patients with extrusion showed higher VEGF concentrations than those of patients in which extrusion was not radiologically established (140 pg/h/mio. cells vs. 106 pg/h/mio. cells).

In degeneration of the intervertebral disc, blood vessels often grow into tissue that is not vascularized in a healthy state. VEGF plays a role in these processes. This suggests a connection between degeneration of the intervertebral disc and VEGF production of the intervertebral disc cells. VEGF can therefore be used as a measure or marker for the cell quality of intervertebral disc cells.

2.4 Correlations with the Modic Score/Modic Changes (Central Intervertebral Disc):

Modic changes refer to changes in the bony structures of the vertebrae.

A further significant correlation was found between the Modic score (central intervertebral disc) and the concentration of MMP-3 in the supernatant of the patient cell cultures. There is a negative correlation between the Modic score and the MMP-3 concentration. This means that patients with a higher Modic score (here, all with score 2; mean MMP-3 concentration: 384 pg/h/mio. cells) showed lower concentrations of MMP-3 in their cell cultures than the patients without Modic changes (score 0; mean MMP-3 concentration: 859 pg/h/mio. cells). In this case, Modic score 0 means that no changes in the vertebrae were detectable by MRT.

MMP 3 is also used as a quality marker for chondrocytes in vitro, wherein a low concentration of MMP-3 is accompanied by loss of the capacity to form cartilage in vivo. As the above-mentioned correlation with the time of the ReOP was observed, this indicates that the cell quality of patients with Modic changes is poorer than that of patients not yet showing any bony changes in the vertebrae. MMP-3 can therefore also be used as a quality marker for intervertebral disc cells.

This is consistent with the result described in the literature that Modic changes are accompanied by decreased expression of MMP-3 (Ding L et al. Cell Biochem Biophys. 2015 Jan. 7 [Epub ahead of print PMID: 25564357]).

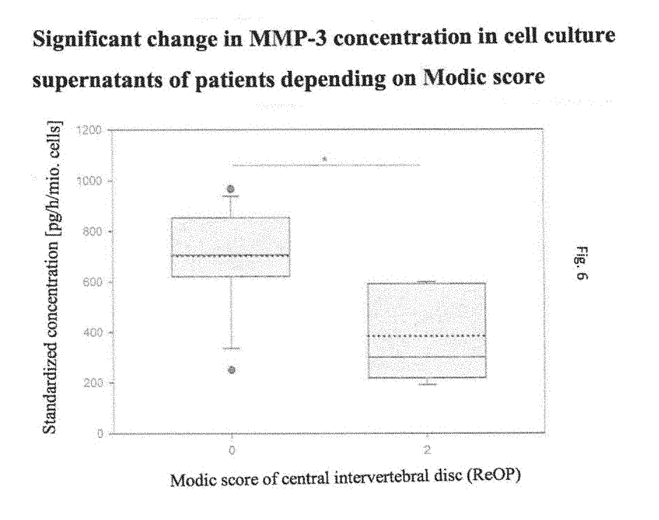

The change in MMP-3 in the cell culture supernatants of the treated patients in relation to the Modic scores is shown in FIG. 6.

2.5 Correlations with the Volume of the Central Intervertebral Disc

Two of the serum markers showed a significant correlation with the volume of the central intervertebral disc. A positive correlation was found for COMP at the time of the ReOP, and a negative correlation for VEGF. This means that the behavior of VEGF concentration in the serum follows a course opposite to that of the volume of the central intervertebral disc, while the behavior of the COMP concentration in the serum and intervertebral disc volume follows the same course. In other words, a higher intervertebral disc volume correlates with a higher COMP concentration and vice versa. The result with respect to VEGF can be attributed to the fact that high VEGF concentrations are accompanied by increased degeneration. Because of the water loss in the intervertebral disc, increased degeneration also means a lower intervertebral disc volume.

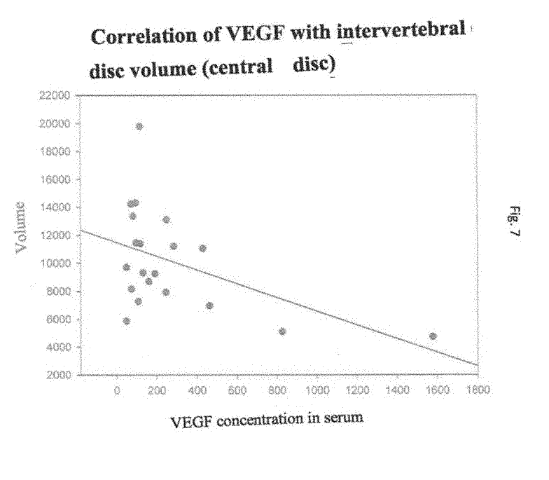

The correlation of VEGF with the volume of the central intervertebral disc is shown in FIG. 7.

Correlations with Modic changes and volume were also observed for hyaluronic acid concentration in the serum of the treated patients.

1.-19. (canceled)

20. A marker for use in vitro in the prediction of a clinical outcome of an autologous disc cell transplantation, in particular a matrix-associated autologous disc cell transplantation, and/or in the progress assessment/progress monitoring of an autologous disc cell transplantation, in particular a matrix-associated autologous disc cell transplantation, and/or in the assessment of the quality of intervertebral disc cells, preferably for an autologous disc cell transplantation, in particular for a matrix-associated autologous disc cell transplantation, and/or in the assessment of the quality of an implant and/or advanced therapy medicinal products for the treatment of an intervertebral disc defect and/or for the reconstruction of intervertebral disc tissue and/or in the diagnosis of an intervertebral disc defect, in particular a lumbar, preferably lumbar-sacral, intervertebral disc defect, and/or spinal column defect, characterized in that the marker is selected from the group consisting of CTX-I, CTX-II, NTX-I, CPII, VEGF, C2C, MMP-3, hyaluronic acid, TNF-alpha, IL-1ra, IL-4, COMP, YKL-40 and combinations of at least two of said markers.

21. The marker of claim 20, characterized in that the marker is a protein in question or proteins in question.

22. The marker of claim 20, characterized in that the marker concentration is determined in body samples taken from a patient at different times.

23. The marker of claim 22, characterized in that an increase or decrease in the concentration of the marker over time is tested.

24. The marker of claim 22, characterized in that the body samples comprise:
a) at least one body sample taken from the patient prior to a sequestrectomy; and/or
b) at least one body sample taken from the patient after sequestrectomy and before an autologous disc cell transplantation, implant-based intervertebral disc treatment, or advanced therapy medicinal product-based intervertebral disc treatment; and/or
c) at least one body sample taken from the patient after an autologous disc cell transplantation, implant-based intervertebral disc treatment, or advanced therapy medicinal product-based intervertebral disc treatment.

25. The marker of claim 22, characterized in that the body samples comprise body samples taken from the patient over a period of up to 24 months after an autologous disc cell transplantation, implant-based intervertebral disc treatment, or advanced therapy medicinal product-based intervertebral disc treatment.

26. The marker of claim 22, characterized in that the body samples are serum samples, plasma samples, urine samples, intervertebral-disc-cell-containing samples or combinations of two or more of said body sample types.

27. The marker of claim 22, characterized in that a decrease in the concentration of CTX-I over time is correlated with the prognosis of a positive clinical outcome and/or with a positive progress assessment/progress monitoring and/or with a positive assessment of the quality of an implant and/or advanced therapy medicinal products.

28. The marker of claim 22, characterized in that a decrease in the concentration of CTX-II over time is correlated with the prognosis of a positive clinical outcome and/or with a positive progress assessment/progress monitoring and/or with a positive assessment of the quality of an implant and/or advanced therapy medicinal products.

29. The marker of claim 22, characterized in that a decrease in the concentration of NTX-I over time is correlated with the prognosis of a positive clinical outcome and/or with a positive progress assessment/progress monitoring and/or with a positive assessment of the quality of an implant and/or advanced therapy medicinal products.

30. The marker of claim 22, characterized in that an increase in the concentration of CPII over time is correlated with the prognosis of a positive clinical outcome and/or with a positive progress assessment/progress monitoring and/or with a positive assessment of the quality of an implant and/or advanced therapy medicinal products.

31. The marker of claim 22, characterized in that an increase in the concentration of C2C over time is correlated with the prognosis of a negative clinical outcome and/or with a negative progress assessment/progress monitoring and/or with a negative assessment of the quality of an implant and/or advanced therapy medicinal products.

32. The marker of claim 20, characterized in that the concentration of the marker in the supernatant of a cell culture containing autologous intervertebral disc cells is determined.

33. The marker of claim 32, characterized in that the cell culture supernatant is tested for an elevated concentration of VEGF, and an elevated concentration of VEGF is correlated with the presence of an intervertebral disc and/or spinal column defect and/or a negative assessment of the quality of the intervertebral disc cells.

34. The marker of claim 32, characterized in that the cell culture supernatant is tested for a decreased concentration of MMP-3, and a decreased concentration of MMP-3 is correlated with the presence of an intervertebral disc and/or spinal column defect and/or a negative assessment of the quality of the intervertebral disc cells.

35. An in vitro method for the prediction of a clinical outcome of an autologous disc cell transplantation, in particular a matrix-associated autologous disc cell transplantation, and/or progress assessment/progress monitoring of an autologous disc cell transplantation, in particular a matrix-associated autologous disc cell transplantation, and/or assessment of the quality of intervertebral disc cells, preferably for an autologous disc cell transplantation, in particular for a matrix-associated autologous disc cell transplantation, and/or assessment of the quality of an implant and/or advanced therapy medicinal products for the treatment of an intervertebral disc defect and/or for the reconstruction of intervertebral disc tissue and/or diagnosis of an intervertebral disc defect, in particular a lumbar, preferably lumbar-sacral, intervertebral disc defect, and/or spinal column defect, characterized in that a marker is used that is selected from the group consisting of CTX-I, CTX-II, NTX-I, CPII, VEGF, C2C, MMP-3, hyaluronic acid, TNF-alpha, IL-1ra, IL-4, COMP, YKL-40 and combinations of at least two of said markers.

36. An in vitro use of a marker for the prediction of a clinical outcome of an autologous disc cell transplantation, in particular a matrix-associated autologous disc cell transplantation, and/or progress assessment/progress monitoring of an autologous disc cell transplantation, in particular a matrix-associated autologous disc cell transplantation, and/or assessment of the quality of intervertebral disc cells, preferably for an autologous disc cell transplantation, in particular for a matrix-associated autologous disc cell transplantation, and/or assessment of the quality of an implant and/or advanced therapy medicinal products for the treatment of an intervertebral disc defect and/or for the reconstruction of intervertebral disc tissue and/or diagnosis of an intervertebral disc defect, in particular a lumbar, preferably lumbar-sacral, intervertebral disc defect, and/or spinal column defect, characterized in that the marker is selected from the group consisting of CTX-I, CTX-II, NTX-I, CPII, VEGF, C2C, MMP-3, hyaluronic acid, TNF-alpha, IL-1ra, IL-4, COMP, YKL-40 and combinations of at least two of said markers.

37. Use of a kit for performing the method of claim 35, characterized in that the kit has at least one substance and/or at least one agent for determining the concentration of the marker.
